(19) United States
(12) Patent Application Publication
(10) Pub. No.: US 2003/0073924 A1
(43) Pub. Date: Apr. 17, 2003

(54) Body fat measuring device

(76) Inventors: Shu-Mei Wu, Taipei (TW); Yao Ou-Yang, Taipei (TW); Chao-Wang Chen, Taipei (TW)

Correspondence Address: RABIN & CHAMPAGNE, P.C., Suite 500, 1101 14 Street, N.W., Washington, DC, 20005, US

(21) Appl. No.: 09/977,378

(22) Filed: Oct. 16, 2001

(51) Int. Cl. A61B 5/05

(52) U.S. Cl. 600/547

(57) ABSTRACT

A card type body fat measuring device provided with only one pair of conductive electrodes being contacted to each one finger of both hands and further cooperated a multi-frequency power source for measurement is disclosed. The present device approaches a frequency-resistance theory to obtain a formula for calculating the measured impedance by an experimental simulation. Regarding to the appearance of the present device, it differs from the conventional one with two pairs of electrodes. Furthermore, the present device has neat circuit layout design and a chipset assembling main circuit units, which is based on the two electrodes and multi-frequency measuring to provide an accurate measurement and a cost-effective production.

### CROSS-REFERENCE TO RELATED APPLICATION

This application makes reference to and incorporates with Taiwan patent application Serial No. 90211630, Filed on Jul. 11, 2001.

### BACKGROUND OF THE INVENTION

1. Field of Invention

The present invention relates in general to a body fat measuring device provided with only one pair of conductive electrodes being contacted to each one finger of both hands for measurement.

2. Description of Related Art

For a body fat determining device, conventionally there has been proposed a method for estimating body density by measuring body impedance between extremities of the body and estimating the body density from the measured impedance value, height and body weight. In theoretical, the ratio of body water in fat-free tissue of body composition tissue is constant and the specific resistance of the fat-free tissue is constant. Further, the density of fat tissue of body is higher than fat-free tissue. Accordingly, it can obtain the inference body density formula as represented by BD=A−k*weight*impedance/(height)², and obtain correlation between the constant A and the value obtained actually by an underwater body weighing method for determining the constants A and k. Next, it can obtain the body fat formula BF (%)=(C₁/BD−C₂)*100, wherein C₁ and C₂ are coefficients.

Moreover, in a conventional method for measuring living body impedance, there are a group of four electrodes consisting of two current path forming electrodes and two measuring electrodes in a body fat determining device, and it has also been necessary to measure body fat as described above, using a method steps of outputting a sine wave of 50 kHz from an oscillator as a constant-voltage source, converting it to a constant current of 800 microamperes by a voltage/current converter, supplying the current through a pair of electrodes, outputting the voltage value of a voltage drop from another pair of electrodes by a differential amplifier, waveform-shaping, rectifying it, DC-converting it, then, A/D-converting it as digital data, and applying it to a calculator, determining the living body impedance Z by Z=V/I from the living body current I and the terminal voltage V, and measuring the voltage V when the current I is constant, thereby determining the living body impedance.

However, when the above-mentioned theoretical method is implemented in practical device, the measuring electrodes will inevitably influence object impedance because of the noise caused by the contact resistance and etc. thereof, and it is difficult to obtain an accurate value of the living body current I as an accurate constant current, thereby causing error in measuring the living body impedance. Furthermore, in order to get an accurate measurement, practically there is used an instrument amplifier of high resistance implemented in the body fat determining device. In addition, with respect to the power source of high frequency as 50 kHz, the body fat determining device needs to introduce constructed elements of high performance. That means the cost is difficult to be reduced.

### SUMMARY OF THE INVENTION

It is thus an object of the present invention to provide a body fat measuring device with only one pair of electrodes serving as the current path forming electrodes as well as the voltage drop measuring electrodes, and further the device cooperated a multi-frequency power source for measurement. Obviously, regarding with an appearance of the present device, it differs from the conventional one with two pairs of contact electrodes. Furthermore, the present device approaches a frequency-resistance theory to obtain a formula for calculating the measured impedance by an experimental simulation instead of the conventional method without formulation but only by measurement relying on the accurately measuring components furnished in the body fat determining device.

The advantages of the present invention are realized by providing a body fat measuring device including a housing, two contact electrodes, a circuit board, a display device and several buttons. The housing has a card shape and size for mounting the circuit board inside. Two electrodes, the display device and the buttons in connection with the circuit board are furnished to be exposed outside the surface of the housing. It is convenient to operate the buttons to input data such as weight, height, year and gender to be stored in a memory unit on the circuit board. While measuring, one finger of both hands steadily contact to each electrode after a start button is depressed. Quickly in seconds later, a measured body fat result with number in percentage and figure will be shown on the display device. The present body fat measuring device is compatible with conventional card type device in manufacturing except for the main characteristics of the present invention with different electronic components mounted on the circuit board and different design of measuring circuit layout in accordance with only one pair of contact electrodes. That is, with the advantages of compact and potable construction, the body fat measuring device of the present invention can substitute the circuit board with specific electronic components and circuit layout easily to complete a mass production.

The present circuit board is based on a measuring method provided by the present invention utilizing two contact electrodes and the multi-frequency power source to obtain the impedance of a measuring object. Two electrodes are served as one pair of current path forming electrodes. A constant-voltage power source is connected to the electrode pair and can output a voltage value with at least two different frequencies controlled by a chipset of the circuit board. During each operation of different frequency outputting, a voltage meter is used to measure a voltage drop between the electrode pair. The voltage drop measured by the voltage meter is caused by the impedance of the measuring body and the resistances of two electrodes. According to the resistance is inversely with respect to the frequency of voltage and in theoretical, when the frequency of voltage becomes infinite, the resistance of the electrode will be diminished to zero, such that an impedance Z can thus be determined by an experimental simulation.

The simulation can be accomplished by experimental way to obtain a first order equation or other higher order equation such as second, third and etc according to two different frequencies. Of course, it can be simulated in a more complicated formula according to further third, forth and etc frequencies. However, the present invention is not limited by the simulating algorithm but to claim the body fat measuring device with only one pair of electrodes cooperated a multi-frequency voltage source for measurement.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become fully understood from the detailed description given hereinbelow illustration only, and thus are not limitative of the present invention, and wherein:

FIG. 1 is the perspective view of a conventional card type body fat determining device.

FIG. 2 shows a circuit board of the body fat determining device of FIG. 1.

FIG. 3 shows a card type body fat measuring device according to the present invention.

FIG. 4 shows a back view of the card type body fat measuring device with respect to FIG. 3.

FIG. 5 depicts a block diagram of the card type body fat measuring device according a preferred embodiment of the present invention.

FIG. 6 depicts a circuit diagram for measuring the impedances according to FIG. 5.

### DETAILED DESCRIPTION OF THE INVENTION

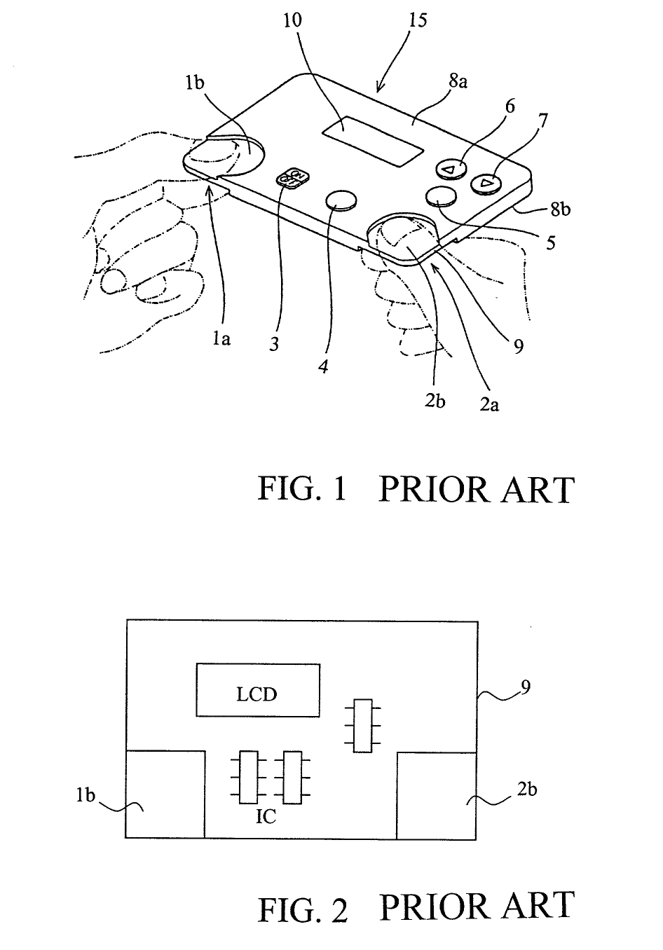

Please referring to FIG. 1 shows a conventional card type body fat determining device 15. A printed circuit board 9, as shown in FIG. 2, is contained inside between a front housing 8a and a rear housing 8b. There are all kinds of necessary electronic components and the circuit layout formed on the circuit board 9 to achieve the measuring function of the device 15.

On the top surface of the body fat determining device 15 has a On/Off button 3 for switching the power and a Selection button 5, a Up button 6 and a Down button 7 for inputting data. The Selection button 5 is operated to choose individual information of inputting data such as height, weight, age, gender and etc. During the operation of the Selection button 5, using alternative Up button 6 or Down button 7 can increase or decrease an input value respectively. In accordance with the combined operations of the Selection, Up and Down buttons 5, 6 and 7, the individual data of a measuring object can thus be inputted.

Further, the body fat determining device 15 has a display portion 10 such as a LCD to show the above-mentioned individual data and a final measuring result of body fat value. A Start button 4 on the surface thereof can then be depressed to actuate a measuring operation.

There are a group of electrodes consisting of four contacts in the body fat determining device 15 for measuring a body impedance value which, alike the above-mentioned individual data, is necessary when obtaining the body fat value. Four contact electrodes form as two pairs including one of a first and a second current path forming electrodes 1a, 2a, and the other of a first and a second measuring electrodes 1b, 2b. In this preferred embodiment, the first and the second current path forming electrodes 1a, 2a are located on the back surface of the body fat determining device 15. Further, the first current path forming electrode 1a is on the right back of the first measuring electrode 1b, and the second current path forming electrode 2a is on the right back of the second measuring electrode 2b.

During the operation, as shown in FIG. 1, the measuring object uses the index finger and thumb of the left hand depressing on the first current path forming electrode 1a and the first measuring electrode 1b, respectively. Similarly, using the index finger and thumb of the right hand depress on the first current path forming electrode 2a and the first measuring electrode 2b, respectively. The impedance of the measuring object can then be determined by well-know measuring mechanism of four electrodes basis described above through contact with electrodes 1a, 1b and 2a, 2b by the operation of the circuit board 9 contained inside the housings 8a and 8b. That is, the circuit board 9 includes the components capable of outputting the constant current, detecting the voltage drop, computing the impedance and etc.

Next, the locations of electrodes 1a and 1b, 2a and 2b are formed as concavities compared with the surfaces of the housing 8a and 8b. Thereby, the fingers can fittingly contact with the electrodes to prevent from a bad measuring result because of varying contact conditions between fingers and electrodes. Such that, it improves the stability and accuracy of the body fat determining device 15.

The electrodes 1a and 1b, 2a and 2b are formed on the circuit board 9 such as a printing circuit board made from epoxy resin. The formations of the electrodes are completed by removing the resist films of the circuit board 9 on the corresponding locations of the electrodes to expose the conductive layers of the circuit board 9. Such that, the conductive layers are directly considered as the electrodes and thus it can be much simpler to build the construction of the body fat determining device 15.

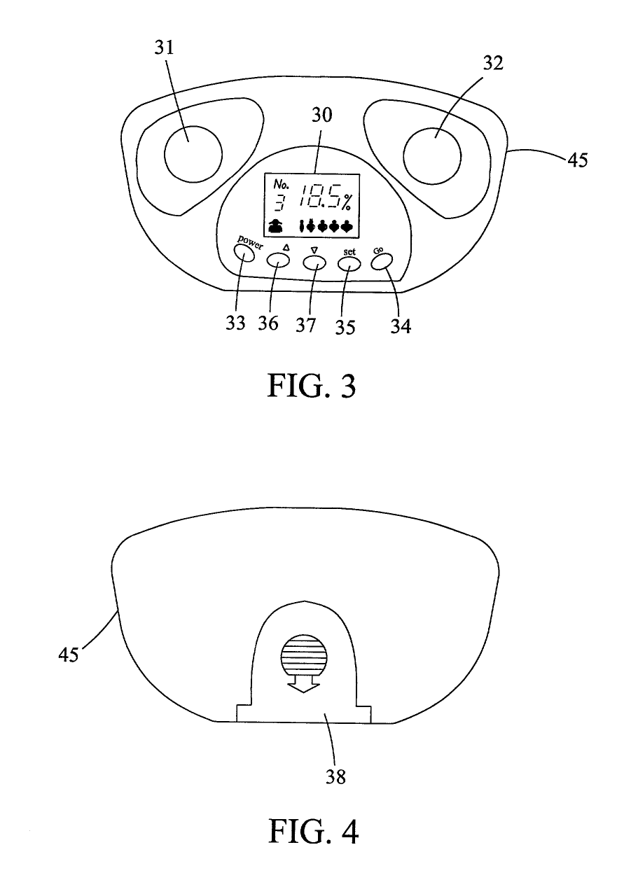

Please now refer to FIG. 3 shown a front view of a body fat measuring device according the present invention. It is obvious to know the differences in appearance by comparison. The body fat measuring device 45 of the present invention only has one pair of electrodes 31, 32, and as shown in FIG. 4, it can find the back surface of the body fat measuring device 45 has no electrodes formed as regarded to the electrodes 1a, 2a of FIG. 1. There is further a battery cover 38 in FIG. 4 to be moved away for putting into the batteries (not shown).

For more explanation, it is not only the appearance with two electrodes 31, 32 differing from the conventional body fat determining device 15. The body fat measuring device 45 of the present invention further provides the multi-frequency measurement with respect to the implement of one-paired electrodes. The following detailed descriptions will show the main characteristics of the present invention.

There are also a pluralities of buttons 33, 34, 35, 36 and 37 corresponding to the above-mentioned On/Off button 3, Start button 4, Selection button 5, and Up and Down buttons 6 and 7 respectively. Similarly, the function of these buttons includes using Power button 33 to connect the power. After the power is on, the LCD monitor 30 will display a formerly recorded individual data. The number 18.5 (represented the measuring result of body fat value) seen on the LCD 30 has not shown yet. Instead, the flashing visible “No. 3” informs the user the present individual data is the third data. It then can perform the Up and Down buttons 36 and 37 to choose a recorded data or to proceed changing the recorded data. Next, the Set button 35 is used to confirm the sequential data such as height, weight, year and gender which is represented by two figures shown on LCD 30 for selection. After the individual information is decided, the Go button 34 is depressed to obtain the measured body fat value (the above-mentioned number 18.5) in seconds and to show one of the figures in five levels to clearly let user know the result.

It is certainly necessary to put one finger of each hand to contact each electrode 31, 32 after the Go button 34 is depressed. For example, in this preferred embodiment, using both thumbs to contact the electrodes 31, 32 are more convenient. However, the present invention is not limited by the locations of the electrodes 31, 32. It depends on the necessities to locate the electrodes 31, 32 on two top corners of the front surface, either on the back surface of the body fat measuring device 45.

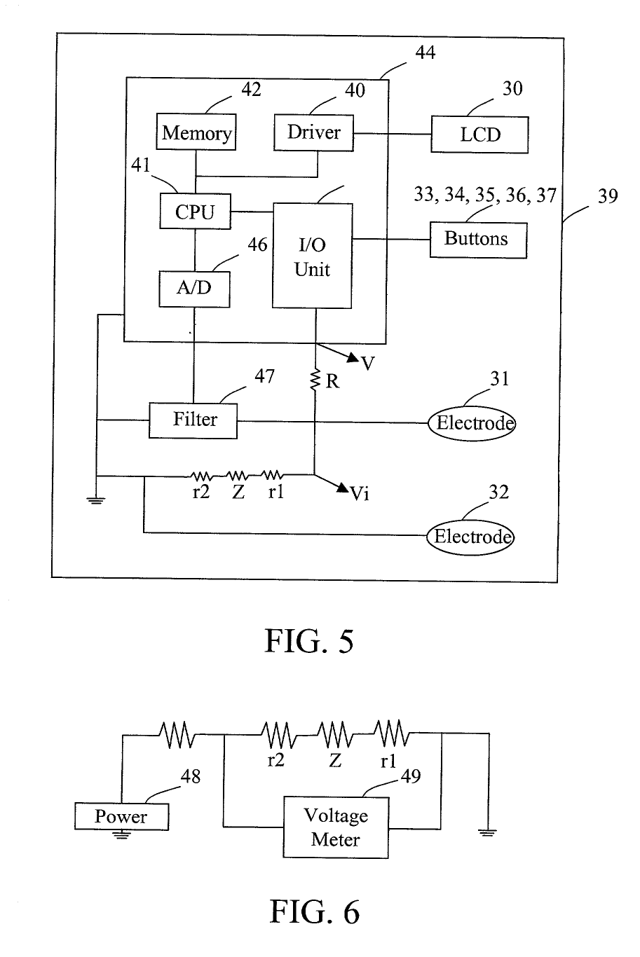

Basically, the body fat measuring device 45 of the present invention can follow other advantages of the conventional body fat determining device 15 in construction. That is, the card type structure provided good portability and mass-producing possibility, which is based on the formation of the electrodes on circuit board, is implemented to the present invention. Please see a block diagram as shown in FIG. 5 for explaining the present circuit board 39 including a central processor unit (CPU) 41, a memory unit 42 and an I/O unit 43. The circuit board 39, of course, has common electronic components such as rectifiers, filters, A/D converters and etc. for measurement; however, those components are belonged to detailed layout diagram so that the present invention does not emphasize on that. As shown in FIG. 5, the block diagram represents the construction of circuit board 39 according to the present invention. Further improvement of the circuit board 39 is to assemble the CPU 41, memory unit 42 and I/O unit 43 in one chipset 44. The circuit layout of the circuit board 39 can thus base on the two electrodes and multi-frequency measuring approach to accomplish a neat and cost-effective design.

Please together refer to FIG. 6, the two electrodes multi-frequency measuring approach of the present invention is as follows. Two electrodes 31, 32 are served as one pair of the current path forming electrodes. A constant-voltage power source 46 connects a resistor R and the electrodes 31, 32 with resistances r1, r2. The power source 46 is controlled by the chipset 44 to output constant voltage of value such as 3 volts with at least two frequencies such as 5 kHz and 20 kHz. Under the operation of a specific voltage V with frequency f, a voltage meter 47 outputs the voltage value of a voltage drop Vi from the electrodes 31, 32 served as one pair of measuring electrodes and then it is determined a impedance Zi consisting of the body impedance and electrode resistances.

That is,

$$Z i = \frac{R * V i}{V - V i}$$

If Z₁ is measured under the first frequency f₁ and Z₂ is measured under the second frequency f₂, according to the resistance is inversely with respect to the frequency of voltage and in theoretical, when the frequency of voltage becomes infinite, the resistance of the electrode will be diminished to zero, such that the body impedance Z can thus be determined by a simulation function as:

Z=Func(Z₁,Z₂, . . . Zᵢ)

If the function Z is simulated in one order equation by two frequencies, it can approaches an equation as:

Z=k₀+k₁Z₁+k₂Z₂

The coefficients of k₀, k₁ and k₂ are determined by experiments. Finally, this equation is used for calculating the body impedance by measurement of Z₁ and Z₂. However, the calculating equation can be approached in other higher order equation such as second, third order etc. according to two different frequencies. Of course, it can be simulated in a more complicated equation according to further third, forth and etc frequencies.

The body fat measuring device 45 of the present invention reduces the electrodes from conventional four electrodes to present two electrodes. Furthermore, the present invention utilizes the simulation under multi-frequencies approach so that expensive instrument amplifier which is the necessary component for the traditional body fat determining device 15 is no more needed. Next, the different circuit layout design of the present invention reduces the necessary circuit components such as the quantities of amplifiers, and the chipset assembles the main components to simplify the circuit design. All of these advantages make the present invention capable of providing an accurate measurement and a cost-effective production.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A body fat measuring device, comprising:
a housing;
two electrodes exposed out of the housing for contacting a measuring object to form a current path;
a plurality of buttons, exposed out of the housing for proceeding an input;
a display device exposed out of the housing for displaying an output; and
a circuit board furnished inside the housing, having a circuit layout to connect the two electrodes, the plurality of buttons and the display, wherein the circuit layout includes a multi-frequency power source for providing the current path a power supply with at least two different frequencies, a meter for measuring a electric value between the two electrodes during the operation of the power supply, and a control chip for controlling and processing the operation of the multi-frequency power source, the meter, the display and the plurality of buttons.

2. The body fat measuring device as claimed in claim 1, wherein the housing has a card shape.

3. The body fat measuring device as claimed in claim 2, wherein the two electrodes are located on the top two corners of the front surface of the housing.

4. The body fat measuring device as claimed in claim 1, wherein the multi-frequency power source is a constant-voltage power source.

5. The body fat measuring device as claimed in claim 1, wherein the meter is a voltage meter.

6. The body fat measuring device as claimed in claim 1, wherein the electric value is a voltage drop value.

7. The body fat measuring device as claimed in claim 1, wherein the at least two different frequencies are 5 kHz and 20 kHz.

8. The body fat measuring device as claimed in claim 1, wherein the control chip comprises a central process unit, a memory unit and an I/O unit.
